(19) United States
(12) Patent Application Publication
(10) Pub. No.: US 2023/0158113 A1
(43) Pub. Date: May 25, 2023

(54) GRANULOCYTE-MACROPHAGE COLONY-STIMULATING FACTOR-BASED INFECTION TREATMENTS

(71) Applicant: Partner Therapeutics, Inc., Lexington, MA (US)

(72) Inventor: Debasish ROYCHOWDHURY, Lexington, MA (US)

(21) Appl. No.: 17/920,075

(22) PCT Filed: Apr. 23, 2021
(86) PCT No.: PCT/US21/28875

§ 371 Date: Oct. 20, 2022

Related U.S. Application Data
(60) Provisional application No. 63/093,576, filed on Oct. 19, 2020.
(60) Provisional application No. 63/014,462, filed on Apr. 23, 2020.

(51) Int. Cl. A61K 38/19; A61K 45/06; A61P 31/14; A61P 11/00

(52) U.S. Cl. CPC A61K 38/193; A61K 45/06; A61P 31/14; A61P 11/00

(57) ABSTRACT

The present disclosure relates to the treatment of infection with coronaviruses with granulocyte-macrophage colony-stimulating factor.

### CROSS-REFERENCE TO RELATED APPLICATIONS

This application claims the benefit of and priority to U.S. Provisional Patent Application No. 63/014,462, filed Apr. 23, 2020, and to U.S. Provisional Patent Application No. 63/093,576, filed Oct. 19, 2020, the content of which are hereby incorporated by reference in their entirety.

### FIELD

This invention relates to, in part, treatment and/or mitigation of a coronavirus infection, including treatment and/or mitigation of an inflammatory cytokine storm.

### SEQUENCE LISTING

The instant application contains a Sequence Listing that has been submitted in ASCII format via EFS-Web and is hereby incorporated by reference in its entirety. Said ASCII copy, created on Apr. 19, 2021, is named “PNR-001PC_ST25.txt” and is 3,951 bytes in size.

### BACKGROUND

Coronaviruses are a family of infective, enveloped, positive-sense, single-stranded RNA viruses that can be divided into four main genres including the alpha, beta, gamma and delta coronaviruses. Fehr A R and Perlman S. Methods Mol Biol 2015; 1282:1-23. In December 2019, one such coronavirus disease was defined as coronavirus disease 19 (COVID-19) and was found to be caused by a novel severe acute respiratory syndrome SARS-CoV-2 virus. SARS-CoV-2 virus (formerly known as 2019-nCoV) is closely related to SARS-CoV, with around 80% identical genome. Zhou P. et al. Nature 2020:10.1038. COVID-19 and SARS-CoV have similar receptor-binding domain (RBD) structures and they use the same cell entry receptor, ACE2. Li W. et al. Nature 2003; 426:450-4.

The RBD domain of the COVID-19 S-protein has a strong interaction with human ACE2 molecules indicating that ACE2 plays an important role in cellular entry, thus ACE2-expressing cells may act as target cells and are susceptible to COVID-19 infection. Li W, et al. Nature 2003; 426:450-4; Xu, X. et al. Science China Life Sciences vol 63, 457-460 (2020). High ACE2 expression has been identified in type II alveolar cells (AT2) of lung, esophagus upper and stratified epithelial cells, absorptive enterocytes from ileum and colon, cholangiocytes, myocardial cells, kidney proximal tubule cells, and bladder urothelial cells. Xu, X. et al. Sci. China Life Sci. vol 63, 457-460 (2020); Zou, X. et al. Front Med. 2020 Mar. 12. doi: 10.1007/s11684-020-0754-0; Zhao, Y. et al. Clin Infect Dis. 2020 Mar. 28. pii: ciaa344. doi: 10.1093/cid/ciaa344; Hao, Xu. et al. International J Oral Science (2020) 12:8; Zhang, H. et al. Cell 181, Apr. 16, 2020; Chai, X. et al. 2020 bioRxiv. doi: 10.1101/2020.02.03.931766.

Many, if not all, viruses, including the respiratory viruses such as influenza, respiratory syncytial virus, rhinovirus and coronavirus, suppress innate immune responses to gain a window of opportunity for efficient virus replication and establishment of the infection. The consequences for the host's immune response are that it is often incomplete, delayed or diminished, or displays overly strong induction (after the delay) that may cause tissue damage. Kikkert M et al. J Innate Immun. 2020 January; 12(1): 4-20. Seemingly mild cases of COVID-19 have been shown to rapidly worsen into severe cases that involve the lower lungs. It is thought that the rapid worsening of COVID-19 cases may be, in part, attributable to “the cytokine storm”, which is an overproduction of immune cells and their activating compounds—cytokines or chemokines—and is often associated with a surge of activated immune cells into the lungs and a systemic inflammatory insult involving many organs. In the lung, the resulting lung inflammation and edema (fluid buildup) can lead to respiratory distress and can be worsened by a secondary bacterial pneumonia, which increases the risk of mortality in patients. These patients can progress rapidly with acute respiratory distress syndrome (ARDS) and septic shock, followed by multiple organ failure. Identification and treatment of this hyperinflammation is important to reduce the rising mortality. Mehta et al. Lancet. 2020 Mar. 28; 395(10229):1033-1034. Most patients present with fever, dry cough, dyspnea, and bilateral ground-glass opacities on chest CT scans. Reports from COVID-19 patients has shown that high amounts of IL-1β, IFNγ, IP10, and MCP1 may be leading to activated T-helper-1 (Th1) cell responses. Critical COVID-19 patients also initiated increased secretion of T-helper-2 (Th2) cytokines (e.g., IL-4 and IL-10), probably as a counter measure to suppresses inflammation, further associating the cytokine storm with disease severity. Additionally, laboratory results from the patients have also identified an increase in IL-6 among other cytokines as a risk factor of cytokine storm in COVID-19-infected pneumonia patients. Cheung C Y et al. 2005. J Virol 79(12):7819-7826; Huang C, et al. Lancet 2020; 395: 497-506; Chen N, et al. Lancet 2020; 395: 507-13; Ruan et al. Intensive Care Med. 2020 Mar. 3. doi: 10.1007/s00134-020-05991-x.

These observations demonstrate a complex cytokine response that builds in viral infection that is characterized by series of overlapping networks. Cytokines TNF and IL-1β and the chemotactic cytokines IL-8 and MCP-1 are indicative of an acute response that appear almost immediately after infection, followed by a more sustained increase in IL-6. Interactions between IL-6 and its soluble receptor enhance the activity of IL-6 on target cells to further aggravate inflammation. Park W Y, et al. 2001. Am. J. Respir. Crit. Care Med. 164:1896-1903.

Compensatory repair processes are initiated soon after inflammation begins, in an attempt to restore tissue and organ function. Systemic production of IL-10 following the onset of a cytokine storm can serve as a marker of suppressive anti-inflammatory response that has been termed “immunoparalysis”, in that it is associated with downregulation of neutrophil and monocyte function in the systemic circulation. Downregulation of systemic inflammation might be conceptually beneficial in controlling systemic responses to local infections. However, it has been suggested that patients who survive the initial cytokine storm but subsequently die are those who do not recover from immunosuppression. Cohen J. 2002. Nature 420:885-891; Fowler A A, et al. Am. J. Pathol. 116:427-435; Tisonick J R. et al. Into the Eye of the Cytokine Storm. Microbiol Mol Biol Rev. 2012 March; 76(1):16-32, Munford R S, et al. 2001. Am. J. Respir. Crit. Care Med. 163:316-321.

Colony Stimulating Factor, CSF, refers to a family of four glycoproteins that control and coordinate cell production by widely scattered deposits of marrow cells. These include: Granulocyte-Macrophage CSF (GM-CSF), Granulocyte colony CSF (G-CSF), Macrophage colony CSF (M-CSF) and multipotential colony-stimulating factor (IL-3). These lymphokines can induce progenitor cells found in the bone marrow to differentiate into specific types of mature blood cells. The particular type of mature blood cell that results from a progenitor cell depends upon the type of CSF present. See Metcalf D. Cancer Immunol Res. 2013, 1(6): 351-356.

GM-CSF is a hematologic growth factor that regulates the production, migration, proliferation, differentiation and function of hematopoietic cells. In response to inflammatory stimuli, GM-CSF is released by various cell types including T lymphocytes, macrophages, fibroblasts and endothelial cells. GM-CSF then activates and enhances the production and survival of neutrophils, eosinophils, and macrophages. Native GM-CSF is usually produced near the site of action where it can modulate proliferation, differentiation, and survival of hematopoietic progenitor cells. It is present in circulating blood in only picomolar concentrations (10⁻¹⁰ to 10⁻¹² M). See Alexander W S. Int Rev Immunol. 1998, 16:651-682; Gasson J C. Blood. 1991, 77:1131-1145; Shannon M F et al. Crit Rev Immunol. 1997, 17:301-323, Barreda D R et al. Dev Comp Immunol. 2004, 28:509-554 and Metcalf D. Immunol Cell Biology. 1987, 65:35-43.

Recombinant human granulocyte-macrophage colony-stimulating factor (rhGM-CSF) has been approved by the FDA for the treatment of neutropenia, blood dyscrasias and malignancies like leukemia in combination with chemotherapies. In the clinic, GM-CSF used for treatment of neutropenia and aplastic anemia following chemotherapy greatly reduces the risk of infection associated with bone marrow transplantation. Its utility in myeloid leukemia treatment and as a vaccine adjuvant is also well established. See Dorr R T. Clin Therapeutics. 1993. 15(1):19-29; Armitage JO. Blood 1998, 92:4491-4508; Kovacic J C et al. J Mol Cell Cardiol. 2007, 42:19-33; Jacobs P P et al. Microbial Cell Factories 2010, 9:93.

Although there are five classes of heterologous protein production platforms, including bacteria, yeasts, plants, insect cells, and mammalian cells, more than 50% of currently marketed biopharmaceuticals are produced in mammalian cell lines. This is in part due to the inability of the remaining four classes to modify glycoproteins with human-like oligosaccharides. This is of importance since protein-bound glycans influence circulation half-life, tissue distribution, biological activity and immunogenicity. LEUKINE is a yeast-derived recombinant humanized granulocyte-macrophage colony stimulating factor (rhuGM-CSF, sargramostim) and the only FDA approved GM-CSF.

Alveolar macrophages (AM) are the first line of host defense against respiratory microbes. GM-CSF is a critical cytokine to help maintain healthy lungs. It contributes to maturation of mononuclear phagocytes and AM. AM from GM-CSF-deficient (GM^{-/-}) mice have impaired capacity for phagocytosis and cytokine production, and these functions were restored by GM-CSF. Studies in GM^{-/-} mice have shown that GM-CSF contributes to immune responses during pneumonia due to Pseudomonas aeruginosa and Pneumocystis carinii, and administration of GM-CSF to septic patients reversed monocyte immunosuppression and improved their clinical course. Further, GM-CSF has been shown to confer resistance to influenza by enhancing innate immune mechanisms that depend on alveolar macrophages. Human recombinant GM-CSF has also been shown to protect against lethal influenza infection in mice. Paine R 3rd et al. J Immunol. 2000. 164(5):2602-9; Paine R 3rd, et al. Am J Physiol Lung Cell Mol Physiol. 2001. 281(5)11210-8; Shi Y et al. Cell Res. 2006. 16(2):126-33; Ballinger M N et al. Am J Respir Cell Mol Biol. 2006. 34(6):766-74; Meisel C et al. Am J Respir Crit Care Med 2009; 180:640-648; Min L et al. J Immunol. 2010. 184(9):4625-9; Huang F-F et al. Am J Respir Crit Care Med. 2011.15; 184(2): 259-268.

Given the pluripotent nature of GM-CSF, several studies have suggested that blocking GM-CSF might inhibit cytokine storm syndrome. Spath S et al. Immunity 46, 245-260; Sterner R M et al. Blood. 2019 Feb. 14; 133(7):697-709; Zhou Y et al. Perspective Immunol. 2020. I-Mab Biopharma has announced plans to develop TJM2 (TJ003234) for the treatment of cytokine storm caused due to severe and critical COVID-19 infection. TJM2 is an antibody that neutralizes human granulocyte-macrophage colony-stimulating factor (GM-CSF). (https://www.pharmaceutical-technology.com/news/i-mab-covid-19-cytokine-storm-therapy/). Interestingly, in a randomized phase II trial, GM-CSF treatment did not increase the number of ventilator free days in patients with acute lung injury (ALI) or ARDS. Paine R 3^{rd}. Crit Care Med. 2012 January; 40(1): 90-97. Accordingly, at least some of the literature suggests that GM-CSF is either ineffective or even deleterious in the context of cytokine storm or lung impairments.

Current management of COVID-19 is supportive, and respiratory failure from ARDS, often paired with cytokine storm, is the leading cause of mortality. There is no approved treatment for COVID-19, and vaccines are being used but their duration and applicability to variants is unknown. Accordingly, there is an urgent need for therapies targeting SARS-CoV-2. There is further need for therapies that could prevent and/or reverse respiratory distress and/or a cytokine storm associated with a coronavirus infection.

### SUMMARY

Accordingly, in one aspect, the present invention relates to a method for treating an infection with a coronavirus, comprising: administering an effective amount of a composition comprising granulocyte-macrophage colony-stimulating factor (GM-CSF) to a patient in need thereof.

In another aspect, the present invention relates to a method for treating an infection with a coronavirus, comprising: administering an effective amount of a composition comprising granulocyte-macrophage colony-stimulating factor (GM-CSF) to a patient in need thereof, wherein the patient is characterized by a reduced number of eosinophils relative to an uninfected state.

In still another aspect, the present invention provides a method for treating an infection with a coronavirus, comprising: (a) selecting a patient having an infection with a coronavirus and one or more of (i) reduced numbers of eosinophils relative to an uninfected state; (ii) elevated level of ferritin relative to an uninfected state; and/or (iii) elevated level of CRP relative to an uninfected state and (b) administering an effective amount of a composition comprising GM-CSF to the patient.

### BRIEF DESCRIPTION OF THE DRAWINGS

FIG. 1 depicts a randomized, open label clinical trial design.

FIG. 2 depicts the absolute change in Alveolar-arterial (A-a) gradient in COVID-19 patients following treatment with LEUKINE.

FIG. 3 depicts the absolute eosinophil count in COVID-19 patients following treatment with LEUKINE.

FIG. 4 depicts the ferritin levels in COVID-19 patients following treatment with LEUKINE.

FIG. 5 depicts the CRP levels in COVID-19 patients following treatment with LEUKINE.

FIG. 6 depicts the lymphocytic count following treatment with LEUKINE.

FIG. 7 depicts the levels of anti-SARS-CoV2 specific immunoglobulins following treatment with LEUKINE. The effect of inhaled LEUKINE on SARS-Cov2 specific immunoglobulins directed against spike (S1) and nucleocapsid (NCV) proteins is shown

FIG. 8 depicts the HLA-DR+CD38+CD8+ T cell count following treatment with LEUKINE. The effect of inhaled LEUKINE on the number of HLA-DR+CD38+CD8 T cells among PBMCs is shown.

FIG. 9 depicts the activated CD8+ T cell count following treatment with LEUKINE. The effect of inhaled LEUKINE on the number of IFNg+ and IL-2+ double positive CD8 T cells is shown.

### DETAILED DESCRIPTION

The present invention relates to, in part, the surprising finding that granulocyte-macrophage colony-stimulating factor (GM-CSF) is an effective agent for coronavirus infections, such as SARS and COVID-19, including, for example, treating and/or reversing a cytokine storm associated with a coronavirus infection and modulating levels of immune cells.

Accordingly, in one aspect, the invention provides methods for treating an infection with a coronavirus.

### Coronaviruses

The coronavirus is a member of the family Coronaviridae, including betacoronavirus and alphacoronavirus respiratory pathogens that have relatively recently become known to invade humans. The Coronaviridae family includes such betacoronavirus as Severe acute respiratory syndrome coronavirus 2 (SARS-CoV-2), SARS-CoV, Middle East Respiratory Syndrome-Corona Virus (MERS-CoV), HCoV-HKU1, and HCoV-0C43. Alphacoronavirus includes, e.g., HCoV-NL63 and HCoV-229E.

Coronaviruses invade cells through “spike” surface glycoprotein that is responsible for viral recognition of Angiotensin Converting Enzyme 2 (ACE2), a transmembrane receptor on mammalian hosts that facilitate viral entrance into host cells. Zhou et al., A pneumonia outbreak associated with a new coronavirus of probable bat origin. Nature 2020. A new coronavirus infection 2019 (COVID-19), caused by

SARS-CoV-2 is a new virus thought to be originated from the bat. COVID-19 causes severe respiratory distress and this RNA virus strain has been the cause of the recent outbreak that has been declared a major threat to public health and worldwide emergency. Phylogenetic analysis of the complete genome of SARS-CoV-2 revealed that the virus was most closely related (89.1% nucleotide similarity) to a group of SARS-like coronaviruses (genus Betacoronavirus, subgenus Sarbecovirus). Wu et al., A new coronavirus associated with human respiratory disease in China. Nature, Feb. 3, 2020.

The SARS-CoV-2 is an enveloped, single stranded, RNA virus that encodes a “spike” protein, also known as the S protein, which is a surface glycoprotein that mediates binding to a cell surface receptor; an integral membrane protein; an envelope protein, and a nucleocapsid protein. The S protein, comprising S1 subunit and S2 subunit, is a trimeric class I fusion protein that exists in a prefusion conformation that undergoes a structural rearrangement to fuse the viral membrane with the host-cell membrane. See, e.g., Li, F. Structure, Function, and Evolution of Coronavirus Spike Proteins. Annu. Rev. Virol. 3: 237-261(2016), which is incorporated herein by reference in its entirety. The structure of the SARS-CoV-2 spike protein in the prefusion conformation has been discovered. See Daniel et al., Cryo-EM structure of the SARS-CoV-2 spike in the prefusion conformation. Science, 19 Feb. 2020, which is incorporated herein by reference in its entirety.

Phylogenetic analysis of the complete genome of SARS-CoV-2 (GenBank Accession No.: MN908947) revealed that the virus was most closely related (89.1% nucleotide similarity) to a group of SARS-like coronaviruses (genus Betacoronavirus, subgenus Sarbecovirus). Wu et al., A new coronavirus associated with human respiratory disease in China. Nature, Feb. 3, 2020, which is incorporated herein by reference in its entirety.

The SARS-CoV-2 has a spike surface glycoprotein, membrane glycoprotein M, envelope protein E, and nucleocapsid phosphoprotein N. The complete genome of the SARS-CoV-2 coronavirus (29903 nucleotides, single-stranded RNA) is described in the NCBI database as GenBank Reference Sequence: MN908947. The coronavirus protein can be selected from the group consisting of: coronavirus spike protein (GenBank Reference Sequence: QHD43416), coronavirus membrane glycoprotein M (GenBank Reference Sequence: QHD43419), coronavirus envelope protein E (GenBank Reference Sequence: QHD43418), and coronavirus nucleocapsid phosphoprotein E (GenBank Reference Sequence: QHD43423).

In embodiments, SARS-CoV-2 coronavirus is the “Wuhan strain” or a variant strain. In some embodiments, the variant strain is one or more of B.1.1.7, B1.351, B.1, B.1.1.28, B.1.2, CAL.20C, B.6, P.1, and P.2 and/or any other variants, or antigenic fragments thereof. In some embodiments, the variant strain is one or more of A.1, A.2, A.3, A.4, A.5, A.6, A.7, A.8, A.9, B, B.1, B.1.1, B.1.1.1, B.2, B.3, B.4, B.5, B.6, B.7, B.9, B.10, B.11, B.12, B.13, B.14, B.15, B.16, B.17, B.18, B.19, B.20, B.21, B.22, B.23, B.24, B.25, B.26, B.27, C.1, C.2, C.3, D.1, and D2. In some embodiments, the variant strain has one or mutations, relative the Wuhan strain, e.g. D614G, E484K, N501Y, K417N, S477G, and S477N.

### Compositions of GM-CSF

GM-CSF used in the practice of the invention includes any pharmaceutically safe and effective GM-CSF, or any derivative thereof having the biological activity of GM-CSF. In an embodiment, the GM-CSF used in the practice of the subject methods is recombinant human GM-CSF (rhu GM-CSF), such as sargramostim (LEUKINE). Sargramostim is a biosynthetic, yeast-derived, recombinant human GM-CSF, having of a single 127 amino acid glycoprotein that differs from endogenous human GM-CSF by having a leucine instead of a proline at position 23. Other natural and synthetic GM-CSFs, and derivatives thereof having the biological activity of natural human GM-CSF, may be equally useful in the practice of the invention.

In embodiments, the GM-CSF is produced or producible in bacteria, yeasts, plants, insect cells, and mammalian cells. In embodiments, the GM-CSF is produced or producible in Escherichia coli cells. In embodiments, the GM-CSF is produced or producible in yeast cells. In embodiments, the GM-CSF is produced or producible in Chinese hamster ovary cells (CHO). In embodiments, the GM-CSF is not produced in E. coli cells. In embodiments, the GM-CSF is produced in a cell that allows for glycosylation, e.g. yeast or CHO cells.

In embodiments, the GM-CSF has an amino acid sequence of SEQ ID NO: 1, or a variant of about 90%, or about 93%, or about 95%, or about 97%, or about 98% identity thereto. In embodiments, the GM-CSF has an amino acid sequence of SEQ ID NO: 2 or SEQ ID NO: 3, or a variant of about 90%, or about 93%, or about 95%, or about 97%, or about 98% identity thereto. In embodiments, the GM-CSF is one of molgramostim, sargramostim, and regramostim.

Without wishing to be bound by theory, the core of hGM-CSF consists of four helices that pack at angles. Crystal structures and mutagenic analysis of rhGM-CSF (Rozwarski D A et al., Proteins 26:304-13, 1996) showed that, in addition to apolar side chains in the protein core, 10 buried hydrogen bonding residues involve intramolecular hydrogen bonding to main chain atoms that were better conserved than residues hydrogen bonding to other side chain atoms; 24 solvation sites were observed at equivalent positions in the two molecules in the asymmetric unit, and the strongest among these was located in clefts between secondary structural elements. Two surface clusters of hydrophobic side chains are located near the expected receptor binding regions. Mutagenesis of residues on the helix A/helix C face confirmed the importance of certain Glu, Gly, and Gln residues. These residues are therefore not to be substituted in the functional substitution variants of hGM-CSF for use in the present invention and these helices are to be retained in a functional fragments or deletion variants of hGM-CSF for use in this invention. Further, in embodiments, one of ordinary skill can reference UniProtKB entry P04141 for structure information to inform the identity of variants.

The N-terminal helix of hGM-CSF governs high affinity binding to its receptor (Shanafelt A B et al., EMBO J 10:4105-12, 1991) Transduction of the biological effects of GM-CSF requires interaction with at least two cell surface receptor components, (one of which is shared with the cytokine IL-5). The above study identified receptor binding determinants in GM-CSF by locating unique receptor binding domains on a series of human-mouse hybrid GM-CSF cytokines. The interaction of GM-CSF with the shared subunit of their high affinity receptor complexes was governed by a very small part of the peptide chains. The presence of a few key residues in the N-terminal α-helix of was sufficient to confer specificity to the interaction.

In some embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile, (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

Modification of the amino acid sequences may be achieved using any known technique in the art e.g., site-directed mutagenesis or PCR based mutagenesis. Such techniques are described, for example, in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989.

Without wishing to be bound by theory, the degree of glycosylation of biosynthetic GM-CSFs appears to influence half-life, distribution, and elimination. (Lieschke and Burgess, N. Engl. J. Med. 327:28-35, 1992; Dorr, R. T., Clin. Ther. 15:19-29, 1993; Horgaard et al., Eur. J. Hematol. 50:32-36, 1993). In embodiments, the present GM-CSF molecules are glycosylated.

### Methods of Treatment

In one aspect, the present invention relates to a method for treating an infection with a coronavirus, comprising: administering an effective amount of a composition comprising granulocyte-macrophage colony-stimulating factor (GM-CSF) to a patient in need thereof.

In another aspect, the present invention relates to a method for treating an infection with a coronavirus, comprising: administering an effective amount of a composition comprising granulocyte-macrophage colony-stimulating factor (GM-CSF) to a patient in need thereof, wherein the patient is characterized by a reduced number of eosinophils relative to an uninfected state.

In another aspect, the present invention provides a method for treating an infection with a coronavirus, comprising: (a) selecting a patient having an infection with a coronavirus and one or more of (i) reduced numbers of eosinophils relative to an uninfected state; (ii) elevated level of ferritin relative to an uninfected state; and/or (iii) elevated level of CRP relative to an uninfected state and (b) administering an effective amount of a composition comprising GM-CSF to the patient.

In embodiments, the method further comprises the step of monitoring eosinophil numbers during the course of treatment. In embodiments, an increased number of eosinophil directs continued administration of GM-CSF. In embodiments, decreased number of eosinophil directs discontinuation of administration of GM-CSF.

In embodiments, wherein the method further comprises the step of monitoring the level of ferritin during the course of treatment. In embodiments, a decreased level of ferritin directs continued administration of GM-CSF. In embodiments, an increased level of ferritin directs discontinuation of administration of GM-CSF.

In embodiments, the method further comprises the step of monitoring the level of CRP during the course of treatment. In embodiments, a decreased level of CRP directs continued administration of GM-CSF. In embodiments, an increased level of CRP directs discontinuation of administration of GM-CSF.

In embodiments, the number of eosinophils, level of ferritin, and/or level of CRP is assayed in a biological sample from the patient.

In embodiments, the coronavirus is selected from: (i) a betacoronavirus, optionally selected from severe acute respiratory syndrome coronavirus 2 (SARS-CoV-2), SARS-CoV, Middle East respiratory syndrome-corona virus (MERS-CoV), HCoV-HKU1, and HCoV-0043 and (ii) an alphacoronavirus, optionally selected from HCoV-NL63 and HCoV-229E. In embodiments, the coronavirus is SARS-CoV-2.

In embodiments, the patient is afflicted with COVID-19. In embodiments, the patient is afflicted with one or more of fever, cough, shortness of breath, diarrhea, upper respiratory symptoms, lower respiratory symptoms, pneumonia, and acute respiratory syndrome. In embodiments, the patient is hypoxic. In embodiments, the patient is afflicted with respiratory distress.

In embodiments, the method increases the number of eosinophils in the patient. In embodiments, the number of eosinophils is assayed in a biological sample from the patient. In embodiments, the biological sample comprises blood, respiratory fluid, saliva, or stool. In embodiments, the respiratory fluid is from an oropharyngeal (OP) or nasopharyngeal (NP) swab. In embodiments, the respiratory fluid is lavage fluid, optionally wherein the lavage fluid comprises a bronchial washing. In embodiments, the respiratory fluid is sputum. In embodiments, the respiratory fluid is a nasal secretion. In embodiments, the respiratory fluid is saliva.

In embodiments, the present methods find use in a patient that is undergoing treatment with one or more steroids, e.g. corticosteroids, e.g. methylprednisone, optionally such steroids being oral inhaled or given by injection. Without wishing to be bound by theory, eosinopenia that induced by steroids can be corrected by the present GM-CSF administration. According, in embodiments, the patient can continue steroid treatment in the context of the present methods.

In embodiments, the method prevents or mitigates development of acute respiratory distress syndrome (ARDS) in the patient. In embodiments, the method improves oxygenation in the patient.

In embodiments, the method causes a decrease in viral load in the patient relative to before treatment.

In embodiments, the method prevents or mitigates a transition from respiratory distress to cytokine imbalance in the patient. In embodiments, the method reverses or prevents a cytokine storm. In embodiments, the method reverses or prevents a cytokine storm in the lungs or systemically. In embodiments, the cytokine storm is selected from one or more of systemic inflammatory response syndrome, cytokine release syndrome, macrophage activation syndrome, and hemophagocytic lymphohistiocytosis. In embodiments, the method reverses or prevents excessive production of one or more inflammatory cytokines. In embodiments, the inflammatory cytokine is one or more of IL-6, IL-1, IL-2, IL-1 receptor antagonist (IL-1ra), IL-2ra, IL-10, IL-18, TNFα, interferon-γ, CXCL10, and CCL7.

In embodiments, the method causes a decrease in ferritin levels relative to before treatment. In embodiments, the method causes a decrease in ferritin to less than about 1000 ng/ml, optionally to less than about 650 ng/ml. In embodiments, the method causes a decrease in C-reactive protein (CRP) relative to before treatment. In embodiments, the method causes a decrease in CRP to less than about 10 mg/L, optionally to less than about 3 mg/L.

In embodiments, the method causes an increase in the quantity and/or quality (e.g. as assayed by anti-infective effectiveness) of SARS-CoV2 antigen-specific, T cells in the patient (e.g. relative to an untreated state or a pre-treated state), e.g. cytotoxic T-cells or helper T cells.

In embodiments, the method causes an increase in the quantity and/or quality (e.g. as assayed by anti-infective effectiveness) of SARS-CoV2 antigen-specific, CD8+ T cells in the patient (e.g. relative to an untreated state or a pre-treated state).

In embodiments, the method causes an increase in the quantity and/or quality (e.g. as assayed by anti-infective effectiveness) of SARS-CoV2 antigen-specific, activated CD8+ T cells in the patient (e.g. relative to an untreated state or a pre-treated state).

In embodiments, the method causes an increase in the quantity and/or quality (e.g. as assayed by anti-infective effectiveness) of SARS-CoV2 antigen-specific, HLD-DR+CD38+CD8+ T cells (e.g. relative to an untreated state or a pre-treated state).

In embodiments, the method causes an increase in the quantity and/or quality (e.g. as assayed by anti-infective effectiveness) of SARS-CoV2 antigen-specific, IFN-g and IL-2 secreting CD8+ T cells (e.g. relative to an untreated state or a pre-treated state).

In embodiments, the method causes a modulation of migratory dendritic cells (DCs). In embodiments, these migratory DCs activate SARS-CoV2 antigen-specific, CD8+ T cells in the patient (e.g. relative to an untreated state or a pre-treated state), e.g. HLD-DR+CD38+CD8+ T cells and/or IFN-g and IL-2 secreting CD8+ T cells.

In embodiments, the method causes a modulation of alveolar macrophages (AMs). In embodiments, these AMs activate SARS-CoV2 antigen-specific, CD8+ T cells in the patient (e.g. relative to an untreated state or a pre-treated state), e.g. HLD-DR+CD38+CD8+ T cells and/or IFN-g and IL-2 secreting CD8+ T cells.

In embodiments, there is provided a method of generating a SARS-CoV2 antigen-specific immune response in a subject by administering the present GM-CSF agents. In embodiments, the SARS-CoV2 antigen-specific immune response is mediated by one or more of CD8+ T cells, AMs, and DCs.

In embodiments, the present GM-CSF enhances an innate immune response, that is modulated AMs, to lead to the present treatment of an infection with a coronavirus.

In embodiments, the present methods provide treatment to a patient who has yet to undergo vaccination or cannot undergo vaccination. In embodiments, the present methods provide treatment to a patient who has undergone vaccination but is infected regardless (e.g. due to ineffective vaccination and/or diminution of the vaccine effect over time). In some embodiments, the coronavirus vaccine comprises one or more of a live attenuated virus, an inactivated virus, a non-replicating viral vector, a replicating viral vector, a recombinant protein, a peptide, a virus-like particle, DNA, RNA, mRNA, another macromolecule, and a fragment thereof. In some embodiments, the coronavirus vaccine is selected from mRNA-1273, AZD1222, BNT162, Ad5-nCoV, INO-4800, and LV-SMENP-DC, and pathogen-specific aAPC, or a variant or derivative thereof. In some embodiments, the coronavirus vaccine comprises an mRNA vaccine encoding SARS-CoV-2 spike (S) protein, optionally LNP-encapsulated, like mRNA-1273. In some embodiments, the coronavirus vaccine comprises a viral vector vaccine expressing the S protein, optionally a viral vector (ChAdOx1—chimpanzee adenovirus Oxford 1) vaccine (ChAdOx1 nCoV-19) expressing the S protein, like AZD1222. In some embodiments, the coronavirus vaccine comprises an mRNA vaccine encoding an optimized SARS-CoV-2 RBD, like BNT162b1. In some embodiments, the coronavirus vaccine comprises an mRNA vaccine encoding an optimized full-length S protein, like BNT162b2. In some embodiments, the coronavirus vaccine comprises Adenovirus type 5 vector that expresses a protein selected from spike surface glycoprotein, membrane glycoprotein M, envelope protein E, and nucleocapsid phosphoprotein N; optionally Adenovirus type 5 vector that expresses S protein, like Ad5-nCoV. In some embodiments, the coronavirus vaccine comprises a plasmid encoding S protein delivered by electroporation, optionally a DNA plasmid encoding S protein delivered by electroporation, like INO-4800. In some embodiments, the coronavirus vaccine comprises dendritic cells (DCs) modified with lentiviral vector expressing synthetic minigene based on domains of selected viral proteins, administered with antigen-specific cytotoxic T lymphocytes (CTLs), like LV-SMENP-DC. In some embodiments, the coronavirus vaccine comprises artificial antigen-presenting cells (aAPCs) modified with lentiviral vector expressing synthetic minigene based on domains of selected viral proteins, like pathogen-specific aAPC.

### Pharmaceutically Acceptable Salts and Excipients

The compositions described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compositions of the present invention having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine, mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In some embodiments, the compositions described herein are in the form of a pharmaceutically acceptable salt.

### Pharmaceutical Compositions and Formulations

In various embodiments, the present invention pertains to pharmaceutical compositions comprising the compositions, e.g. GM-CSF and/or an additional therapeutic agent, described herein and a pharmaceutically acceptable carrier or excipient. Any pharmaceutical compositions described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

In various embodiments, pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

The present invention includes the described pharmaceutical compositions (and/or additional therapeutic agents) in various formulations. Any inventive pharmaceutical composition (and/or additional therapeutic agents) described herein can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, dessicated powder, or any other form suitable for use. In one embodiment, the composition is in the form of a capsule. In another embodiment, the composition is in the form of a tablet. In yet another embodiment, the pharmaceutical composition is formulated in the form of a soft-gel capsule. In a further embodiment, the pharmaceutical composition is formulated in the form of a gelatin capsule. In yet another embodiment, the pharmaceutical composition is formulated as a liquid.

Where necessary, the inventive pharmaceutical compositions (and/or additional therapeutic agents) can also include a solubilizing agent. Also, the agents can be delivered with a suitable vehicle or delivery device as known in the art. Combination therapies outlined herein can be co-delivered in a single delivery vehicle or delivery device.

The formulations comprising the inventive pharmaceutical compositions (and/or additional therapeutic agents) of the present invention may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In various embodiments, any pharmaceutical compositions (and/or additional therapeutic agents) described herein is formulated in accordance with routine procedures as a composition adapted for a mode of administration described herein.

Routes of administration include, for example: oral, intradermal, intramuscular, intraperitoneal, intravenous, subcutaneous, intranasal, epidural, sublingual, intranasal, intracerebral, intravaginal, transdermal, rectally, by inhalation, or topically. Administration can be local or systemic. In some embodiments, the administering is effected orally. In another embodiment, the administration is by parenteral injection. The mode of administration can be left to the discretion of the practitioner, and depends in-part upon the site of the medical condition. In most instances, administration results in the release of any agent described herein into the bloodstream.

In specific embodiments, the GM-CSF (and/or additional therapeutic agents) is administered via an intravenous route.

In specific embodiments, the GM-CSF (and/or additional therapeutic agents) is administered to the lung.

In specific embodiments, the GM-CSF (and/or additional therapeutic agents) is administered via aerosol or nebulizer.

In specific embodiments, the aerosol or nebulizer is selected from liquid nebulization, dry powder dispersion and meter-dose administration. In specific embodiments, the aerosol or nebulizer is selected from jet flow or mesh vibrating.

In specific embodiments, the GM-CSF (and/or additional therapeutic agents) is administered by inhalation. Without wishing to be bound by theory, inhalation of sargramostim produces low systemic exposure in the patient.

In embodiments, the GM-CSF (and/or additional therapeutic agents) is administered by inhalation and mediates local and/or peripheral cellular responses. For instance, in embodiments, the GM-CSF (and/or additional therapeutic agents) is administered by inhalation and mediates an increase in lymphocytes and/or eosinophils in the peripheral blood.

Accordingly, in embodiments, there is provided a method of modulating peripheral immune cells to cause an anti-infective effect through local administration (e.g. inhalation).

In one embodiment, the pharmaceutical compositions (and/or additional therapeutic agents) described herein are formulated in accordance with routine procedures as a composition adapted for oral administration. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any pharmaceutical compositions (and/or additional therapeutic agents) described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

The compositions provided herein, alone or in combination with other suitable components, can be made into aerosol formulations (i.e., “nebulized”) to be administered via inhalation. Aerosol formulations can be placed into pressurized acceptable propellants, such as dichlorodifluoromethane, propane, nitrogen, and the like.

Any inventive pharmaceutical compositions (and/or additional therapeutic agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropyl cellulose, hydropropylmethyl cellulose, polyvinylpyrrolidone, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The invention thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

### Administration and Dosage

It will be appreciated that the actual dose of the composition to be administered according to the present invention will vary according to the particular dosage form, and the mode of administration. Many factors that may modify the action of the composition (e.g., body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

In embodiments, the GM-CSF is administered at a total dose of about 125 μg, about 150 μg, or about 200 μg, or about 250 μg, or about 300 μg, or about 350 μg. In embodiments, the GM-CSF is administered at a total dose of about 250 μg.

In embodiments, the GM-CSF is administered at a dose of about 125 μg, about 150 μg, or about 200 μg, or about 250 μg, or about 300 μg, or about 350 μg.

In embodiments, the GM-CSF is administered twice daily.

In embodiments, the GM-CSF is sargramostim, administered at a dose of about 125 μg, twice daily.

### Combination Therapy and Additional Therapeutic Agents

In various embodiments, the pharmaceutical composition of the present invention is co-administered in conjunction with additional agent(s). Co-administration can be simultaneous or sequential.

In one embodiment, the additional therapeutic agent and the GM-CSF of the present invention are administered to a subject simultaneously. The term “simultaneously” as used herein, means that the additional therapeutic agent and the GM-CSF are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the GM-CSF can be by simultaneous administration of a single formulation (e.g., a formulation comprising the additional therapeutic agent and the GM-CSF composition) or of separate formulations (e.g., a first formulation including the additional therapeutic agent and a second formulation including the GM-CSF composition).

Co-administration does not require the therapeutic agents to be administered simultaneously, if the timing of their administration is such that the pharmacological activities of the additional therapeutic agent and the GM-CSF overlap in time, thereby exerting a combined therapeutic effect. For example, the additional therapeutic agent and the targeting moiety, the GM-CSF composition can be administered sequentially. The term “sequentially” as used herein means that the additional therapeutic agent and the GM-CSF are administered with a time separation of more than about 60 minutes. For example, the time between the sequential administration of the additional therapeutic agent and the GM-CSF can be more than about 60 minutes, more than about 2 hours, more than about 5 hours, more than about 10 hours, more than about 1 day, more than about 2 days, more than about 3 days, more than about 1 week apart, more than about 2 weeks apart, or more than about one month apart. The optimal administration times will depend on the rates of metabolism, excretion, and/or the pharmacodynamic activity of the additional therapeutic agent and the GM-CSF being administered. Either the additional therapeutic agent or the GM-CSF composition may be administered first.

Co-administration also does not require the therapeutic agents to be administered to the subject by the same route of administration. Rather, each therapeutic agent can be administered by any appropriate route, for example, parenterally or non-parenterally.

In some embodiments, the GM-CSF described herein acts synergistically when co-administered with another therapeutic agent. In such embodiments, the targeting moiety, the GM-CSF composition and the additional therapeutic agent may be administered at doses that are lower than the doses employed when the agents are used in the context of monotherapy.

In some embodiments, the additional therapeutic agent is selected from remdesivir; favipiravir; galidesivir; prezcobix; lopinavir; and/or ritonavir; and/or arbidol lopinavir/ritonavir; and/or ribavirin; and/or IFN-beta; xiyanping; anti-VEGF-A; fingolimod; carrimycin; hydroxychloroquine; darunavir and cobicistat; methylprednisolone; brilacidin; leronlimab; and thalidomide.

In some embodiments, the additional therapeutic agent is an antibody, e.g. monoclonal antibody, directed against an antigen of a coronavirus, e.g. the spike protein, e.g. the RBD thereof. In some embodiments, the additional therapeutic agent is one or more of bamlanivimab, casirivimab, and imdevimab.

### Methods of Detecting Therapeutic Effect

In some embodiments, there is provided a method for detecting the likelihood of successful treatment or response to treatment with the present GM-CSF agents for an infection with a coronavirus. In embodiments, there is provided a method for detecting the likelihood of successful treatment or response to treatment with the present GM-CSF agents for an infection with a coronavirus by using KL-6 as a biomarker. In embodiments, the present GM-CSF agents cause a decrease in KL-6 in subjects with coronavirus infection and therefore, this reduction is used as a biomarker for treatment. For instance, in embodiments, KL-6 levels are determined (e.g. as assayed via protein or nucleic acid levels in a biological sample, e.g. blood, respiratory fluid (e.g. from an oropharyngeal (OP) or nasopharyngeal (NP) swab, or a lavage fluid, optionally wherein the lavage fluid comprises a bronchial washing, also including sputum, a nasal secretion, or saliva), saliva, or stool. In embodiments, decreasing KL-6 (e.g. relative to pre-treatment or untreated), is indicative of successful treatment while a lack no decreasing, or increasing, of KL-6 is indicative of less successful treatment (e.g. directing to higher dosing and/or alternative treatments (e.g. one or more of those disclosed herein).

In some embodiments, there is provided a method for detecting the likelihood of successful treatment with the present GM-CSF agents for an infection with a coronavirus, by assaying the presence, absence, or level of one or more MUC1 antigens, including, without limitation KL-6. For instance, in embodiments, there is provided a method of obtaining a sample from a patient who has, or is suspected to have, an infection with a coronavirus and assaying the presence, absence, or level of one or more MUC1 antigens, including, without limitation KL-6, wherein (i) a high level (e.g. relative to normal or uninfected levels) indicates a high likelihood of successful treatment and directs treatment or continued treatment with the present GM-CSF agents or (ii) a low level (e.g. relative to normal or uninfected levels) indicates a low likelihood of successful treatment and directs treatment with an alternative treatment to the present GM-CSF agents.

In some embodiments, there is provided a method for detecting response to treatment with the present GM-CSF agents for an infection with a coronavirus, by assaying the presence, absence, or level of one or more MUC1 antigens, including, without limitation KL-6. For instance, in embodiments, there is provided a method of obtaining a sample from a patient who has, or is suspected to have, an infection with a coronavirus and is receiving treatment with the present GM-CSF agents and assaying the presence, absence, or level of one or more MUC1 antigens, including, without limitation KL-6, wherein (i) a low level (e.g. relative to normal or uninfected levels) indicates a positive response to treatment and directs continued treatment with the present GM-CSF agents or (ii) a high level (e.g. relative to normal or uninfected levels) indicates a negative response to treatment and directs treatment with an alternate agent.

### Uses of Molgramostim

In some embodiments, the present invention pertains to the use of molgramostim in a method of treating or preventing an infection, by administering via inhalation and, optionally, modulating an immune response in the periphery (e.g. via modulating antigen-specific, CD8+ T cells in the patient (e.g. relative to an untreated state or a pre-treated state), e.g. HLD-DR+CD38+CD8+ T cells and/or IFN-g and IL-2 secreting CD8+ T cells).

In embodiments, such molgramostim is used in a method of treating or preventing a viral infection, a parasitic infection, or a bacterial infection.

In various embodiments, the present invention provides methods of treating viral infections with molgramostim, the viral infection being a coronavirus is selected from: (i) a betacoronavirus, optionally selected from severe acute respiratory syndrome coronavirus 2 (SARS-CoV-2), SARS-CoV, Middle East respiratory syndrome-corona virus (MERS-CoV), HCoV-HKU1, and HCoV-0043 and (ii) an alphacoronavirus, optionally selected from HCoV-NL63 and HCoV-229E. In embodiments, the coronavirus is SARS-CoV-2.

In various embodiments, the present invention provides methods of treating viral infections with molgramostim including, without limitation, acute or chronic viral infections, for example, of the respiratory tract, of papilloma virus infections, of herpes simplex virus (HSV) infection, of human immunodeficiency virus (HIV) infection, and of viral infection of internal organs such as infection with hepatitis viruses. In some embodiments, the viral infection is caused by a virus of family Flaviviridae. In some embodiments, the virus of family Flaviviridae is selected from Yellow Fever Virus, West Nile virus, Dengue virus, Japanese Encephalitis Virus, St. Louis Encephalitis Virus, and Hepatitis C Virus. In other embodiments, the viral infection is caused by a virus of family Picornaviridae, e.g., poliovirus, rhinovirus, coxsackievirus. In other embodiments, the viral infection is caused by a member of Orthomyxoviridae, e.g., an influenza virus. In other embodiments, the viral infection is caused by a member of Retroviridae, e.g., a lentivirus. In other embodiments, the viral infection is caused by a member of Paramyxoviridae, e.g., respiratory syncytial virus, a human parainfluenza virus, rubulavirus (e.g., mumps virus), measles virus, and human metapneumovirus. In other embodiments, the viral infection is caused by a member of Bunyaviridae, e.g., hantavirus. In other embodiments, the viral infection is caused by a member of Reoviridae, e.g., a rotavirus.

In various embodiments, the present invention provides methods of treating parasitic infections with molgramostim such as protozoan or helminths infections. In some embodiments, the parasitic infection is by a protozoan parasite. In some embodiments, the oritiziab parasite is selected from intestinal protozoa, tissue protozoa, or blood protozoa. Illustrative protozoan parasites include, but are not limited to, Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Toxoplasma gondii, Plasmodium vivax, Plasmodium ovale, Plasmodium malariae, Plasmodium falciparum, Trichomonas vaginalis, and Histomonas meleagridis. In some embodiments, the parasitic infection is by a helminthic parasite such as nematodes (e.g., Adenophorea). In some embodiments, the parasite is selected from Secementea (e.g., Trichuris trichiura, Ascaris lumbricoides, Enterobius vermicularis, Ancylostoma duodenale, Necator americanus, Strongyloides stercoralis, Wuchereria bancrofti, Dracunculus medinensis). In some embodiments, the parasite is selected from trematodes (e.g. blood flukes, liver flukes, intestinal flukes, and lung flukes). In some embodiments, the parasite is selected from: Schistosoma mansoni, Schistosoma haematobium, Schistosoma japonicum, Fasciola hepatica, Fasciola gigantica, Heterophyes, Paragonimus westermani. In some embodiments, the parasite is selected from cestodes (e.g., Taenia solium, Taenia saginata, Hymenolepis nana, Echinococcus granulosus).

In various embodiments, the present invention provides methods of treating bacterial infections with molgramostim. In various embodiments, the bacterial infection is by a gram-positive bacteria, gram-negative bacteria, aerobic and/or anaerobic bacteria. In various embodiments, the bacteria is selected from, but not limited to, Staphylococcus, Lactobacillus, Streptococcus, Sarcina, Escherichia, Enterobacter, Klebsiella, Pseudomonas, Acinetobacter, Mycobacterium, Proteus, Campylobacter, Citrobacter, Nisseria, Baccillus, Bacteroides, Peptococcus, Clostridium, Salmonella, Shigella, Serratia, Haemophilus, Brucella and other organisms. In some embodiments, the bacteria is selected from, but not limited to, Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

Sequences
SEQ ID NO: 1 is wild type GM-CSF:
APARSPSPSTQPWEHVNAIQEAPRLLNLSRDTAAEMNE
TVEVISEMFDLQEPTCLQTRLELYKQGLRGSLTKLKGP
LTMMASHYKQHCPPTPETSCATQIITFESFKENLKDFL
LVIPFDCWEPVQE.
SEQ ID NO: 2 is sargramostim:
APARSPSPSTQPWEHVNAIQEALRLLNLSRDTAAEMNE
TVEVISEMFDLQEPTCLQTRLELYKQGLRGSLTKLKGP
LTMMASHYKQHCPPTPETSCATQIITFESFKENLKDFL
LVIPFDCWEPVQE.
SEQ ID NO: 3 is molgramostim:
APARSPSPSTQPWEHVNAIQEARRLLNLSRDTAAEMNE
TVEVISEMFDLQEPTCLQTRLELYKQGLRGSLTKLKGP
LTMMASHYKQHCPPTPETSCATQIITFESFKENLKDFL
LVIPFDCWEPVQE.

### Definitions

The following definitions are used in connection with the invention disclosed herein. Unless defined otherwise, all technical and scientific terms used herein have the same meaning as commonly understood to one of skill in the art to which this invention belongs.

An “effective amount,” when used in connection with an agent effective for the treatment of a coronavirus infection is an amount that is effective for treating or mitigating a coronavirus infection.

As used herein, “a,” “an,” or “the” can mean one or more than one. Further, the term “about” when used in connection with a referenced numeric indication means the referenced numeric indication plus or minus up to 10% of that referenced numeric indication. For example, the language “about 50” covers the range of 45 to 55.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

This invention is further illustrated by the following non-limiting examples.

### EXAMPLES

### Example 1: Design of Clinical Trial of Sargramostim (LEUKINE) for Treatment of COVID-19

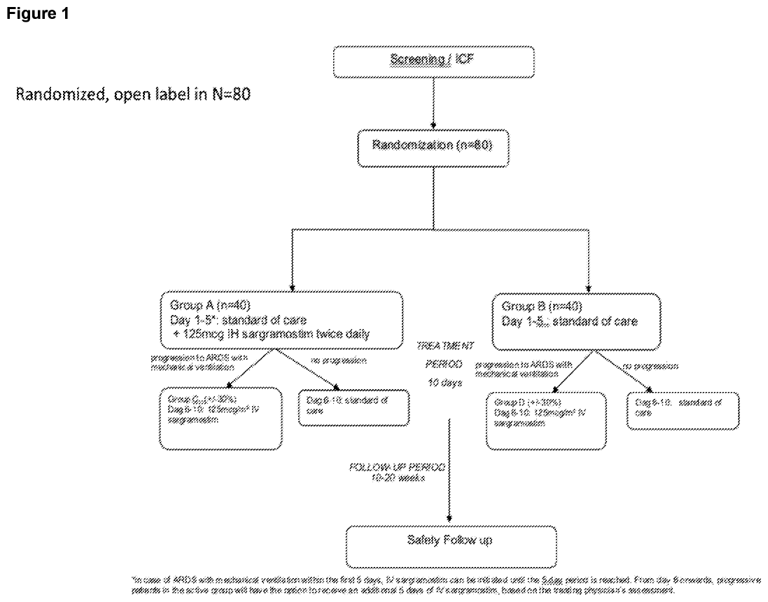

A prospective, randomized, open-label, interventional study to investigate the efficacy of sargramostim (LEUKINE) in improving oxygenation and short- and long-term outcome of COVID-19 patients with acute hypoxic respiratory failure was performed (FIG. 1).

Potential participants, 18-80 years old, were identified when they presented with symptoms of COVID-19 infection that met clinical, radiological and laboratory criteria for ARDS. Patients were divided onto 2 groups: Group A patients received Standard of care+125 μcg inhaled (IH) sargramostim twice daily, and Group B patients received only standard of care (SOC) from days 1-5 (D1-5). After D5, if patients progressed to ARDS with mechanical ventilation, such patients in both groups were given 125 μcg/m² sargramostim intravenously (IV) (Groups C and D respectively). Patients will continue to be monitored for an additional 10-20 weeks following treatment.

Exclusion criteria included patients with known hypersensitivity to human granulocyte-macrophage colony stimulating factor such as sargramostim (GM-CSF), yeast-derived products, or any component of LEUKINE, patients who were enrolled in other clinical trials or pregnant or breastfeeding, patients with peripheral white blood cell count above 25,000 per ml and/or active malignancy, patients on high dose systemic steroids (>20 mg methylprednisolone or equivalent) or lithium carbonate therapy and patients with serum ferritin >2000 μcg/ml.

The primary objective was to investigate whether the administration of inhaled LEUKINE during the first 5 days improves oxygenation in COVID-19 patients. Secondary objectives were to determine if early intervention with LEUKINE had a good safety profile and affected the progression to mechanical ventilation and/or ARDS

### Example 2: Clinical Results

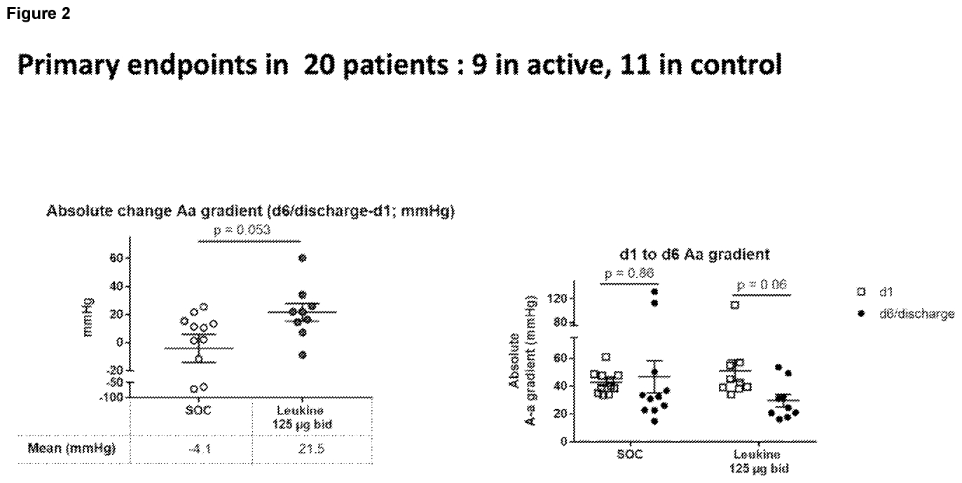

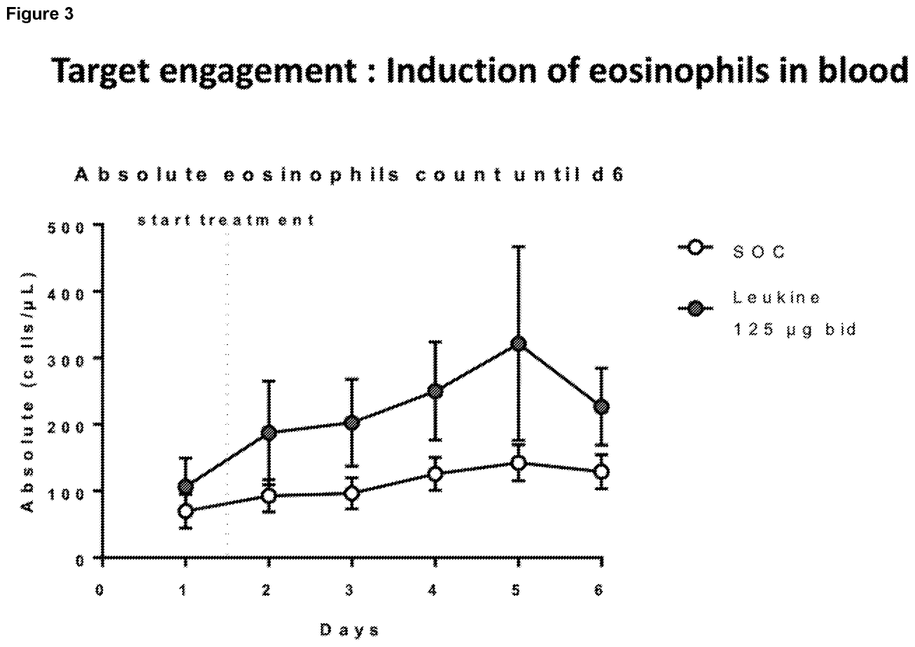

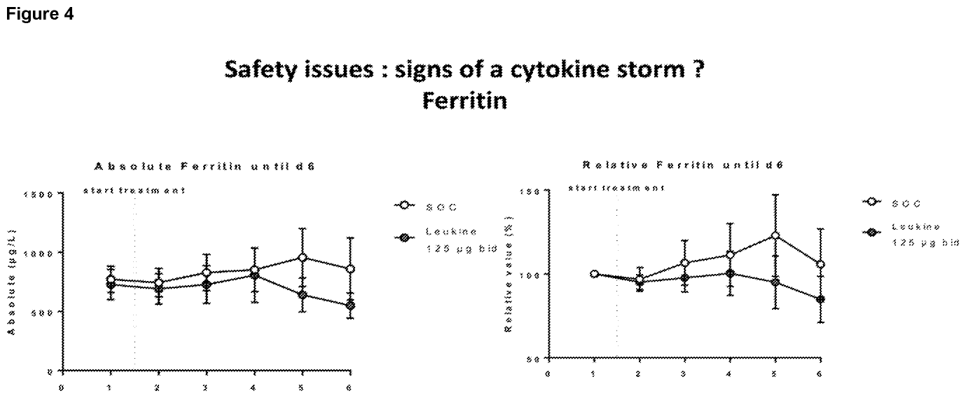

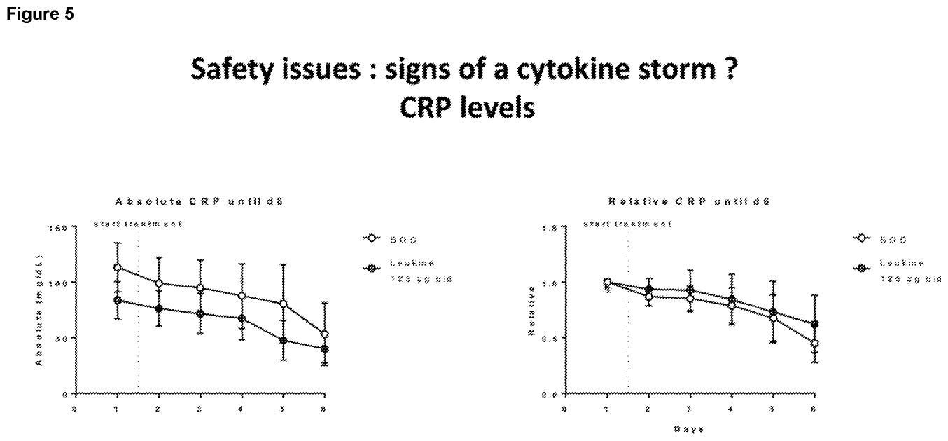

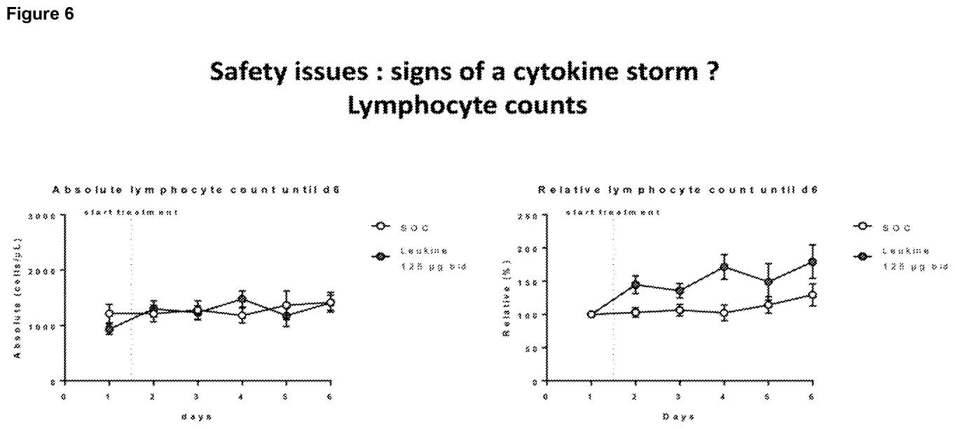

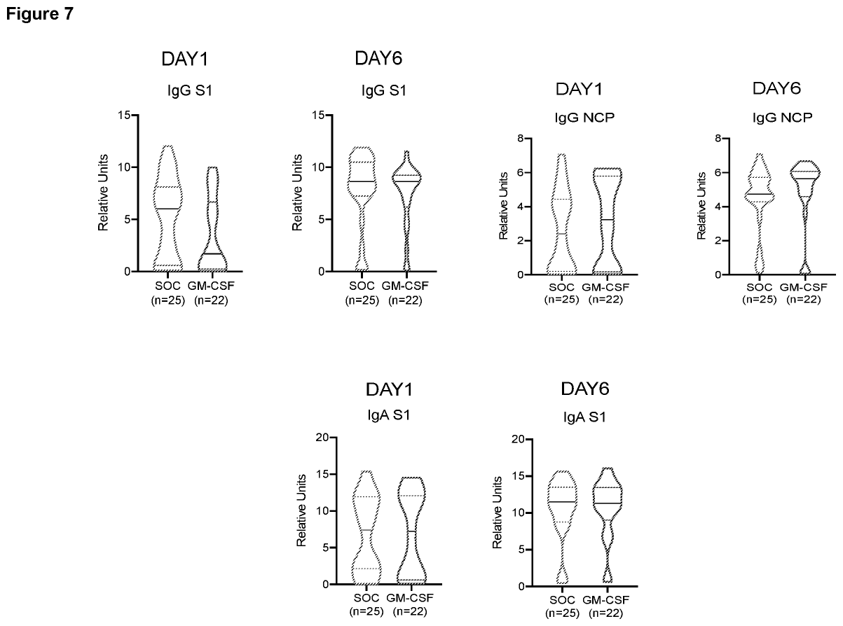

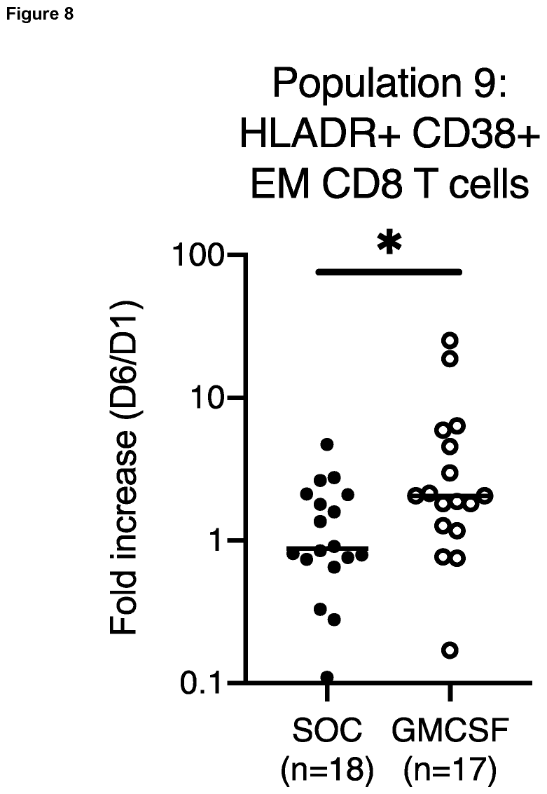

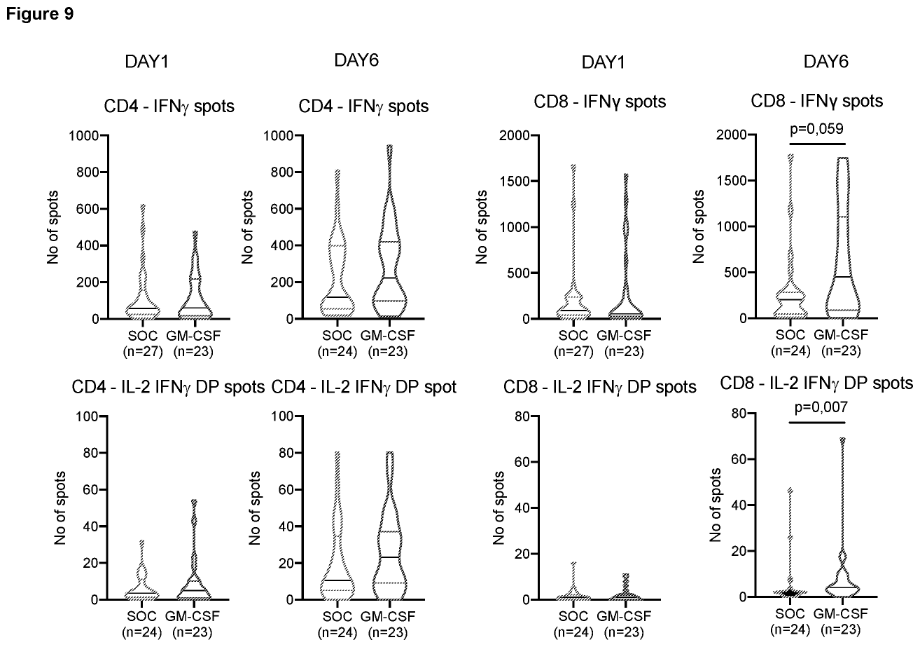

Administration of LEUKINE to COVID-19 infected patients caused a decrease in the absolute Alveolar-arterial (A-a) gradient from D1 to D6 compared to SOC. This suggests that administration of LEUKINE to patients started to normalize any defect in oxygen diffusion into the blood caused by COVID-19 (FIG. 2). Treatment with LEUKINE also increased absolute eosinophil count compared to SOC (FIG. 3). Further and importantly, treatment with LEUKINE decreased ferritin and CRP levels, while maintaining the absolute lymphocytic count in patients compared to SOC (FIGS. 4-6). These can be markers of a cytokine storm and potential toxicity, and thus indicate that LEUKINE can be safely administered to patients. Further assessment of the immune response from D1 to D6 following treatment with LEUKINE, showed a significant increase in the number of SARS-CoV2 antigen-specific, HLD-DR+CD38+CD8+ T cells (FIG. 8), as well as an increase in the number of activated, IFN-g and IL-2 secreting CD8+ T cells (FIG. 9). However, treatment with LEUKINE did not show a remarkable increase in the amount of IgG antibodies directed at the SARS-CoV2 spike (S1) and nucleocapsid (NCV) proteins or IgA antibody directed at the spike (S1) protein measured between D1 and D6 (FIG. 7).

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

As used herein, all headings are simply for organization and are not intended to limit the disclosure in any manner. The content of any individual section may be equally applicable to all sections.

What is claimed is:

1. A method for treating an infection with a coronavirus, comprising: administering an effective amount of a composition comprising granulocyte-macrophage colony-stimulating factor (GM-CSF) to a patient in need thereof.

2. A method for treating an infection with a coronavirus, comprising: administering an effective amount of a composition comprising granulocyte-macrophage colony-stimulating factor (GM-CSF) to a patient in need thereof,
wherein the patient is characterized by a reduced number of eosinophils relative to an uninfected state.

3. The method of claim 1 or 2, wherein the coronavirus is selected from
(i) a betacoronavirus, optionally selected from severe acute respiratory syndrome coronavirus 2 (SARS-CoV-2), SARS-CoV, Middle East respiratory syndrome-corona virus (MERS-CoV), HCoV-HKU1, and HCoV-0043 and
(ii) an alphacoronavirus, optionally selected from HCoV-NL63 and HCoV-229E.

4. The method of claim 1, wherein the coronavirus is SARS-CoV-2 and optionally the patient is afflicted with COVID-19.

5. The method of claim 2, wherein the coronavirus is SARS-CoV-2 and optionally the patient is afflicted with COVID-19.

6. The method of any one of claims 1-5, wherein the patient is afflicted with one or more of fever, cough, shortness of breath, diarrhea, upper respiratory symptoms, lower respiratory symptoms, pneumonia, and acute respiratory syndrome.

7. The method of any one of claims 1-6, wherein the patient is hypoxic.

8. The method of any one of claims 1-7, wherein the patient is afflicted with respiratory distress.

9. The method of any one of claims 1-8, wherein the method increases the number of eosinophils in the patient.

10. The method of claim 9, wherein the number of eosinophils is assayed in a biological sample from the patient.

11. The method of claim 10, wherein the biological sample comprises blood, respiratory fluid, saliva, or stool.

12. The method of claim 11, wherein the respiratory fluid is from an oropharyngeal (OP) or nasopharyngeal (NP) swab.

13. The method of claim 11, wherein the respiratory fluid is lavage fluid, optionally wherein the lavage fluid comprises a bronchial washing.

14. The method of claim 11, wherein the respiratory fluid is sputum.

15. The method of claim 11, wherein the respiratory fluid is a nasal secretion.

16. The method of claim 11, wherein the respiratory fluid is saliva.

17. The method of any one of claims 1-16, wherein the method prevents or mitigates development of acute respiratory distress syndrome (ARDS) in the patient.

18. The method of any one of claims 1-17, wherein the method improves oxygenation in the patient.

19. The method of any one of claims 1-18, wherein the method prevents or mitigates a transition from respiratory distress to cytokine imbalance in the patient.

20. The method of any one of claims 1-19, wherein the method reverses or prevents a cytokine storm.

21. The method of claim 20, wherein the method reverses or prevents a cytokine storm in the lungs or systemically.

22. The method of claim 20 or 21, wherein the cytokine storm is selected from one or more of systemic inflammatory response syndrome, cytokine release syndrome, macrophage activation syndrome, and hemophagocytic lymphohistiocytosis.

23. The method of claim 20 or 21, wherein the method reverses or prevents excessive production of one or more inflammatory cytokines.

24. The method of claim 23, wherein the inflammatory cytokine is one or more of IL-6, IL-1, IL-2, IL-1 receptor antagonist (IL-1ra), IL-2ra, IL-10, IL-18, TNFα, interferon-γ, CXCL10, and CCL7.

25. The method of any one of claims 1-24, wherein the method causes a decrease in viral load in the patient relative to before treatment.

26. The method of any one of claims 1-25, wherein the method causes a decrease in ferritin levels relative to before treatment.

27. The method of claim 26, wherein the method causes a decrease in ferritin to less than about 1000 ng/ml, optionally to less than about 650 ng/ml.

28. The method of any one of claims 1-27, wherein the method causes a decrease in C-reactive protein (CRP) relative to before treatment, optionally a decrease in CRP to less than about 10 mg/L, optionally to less than about 3 mg/L.

29. The method of claim 28, wherein the method causes an increase in HLD-DR+CD38+CD8+ T cells in the patient.

30. The method of any one of claims 1-29, wherein the GM-CSF has an amino acid sequence of SEQ ID NO: 1, or a variant of about 90%, or about 93%, or about 95%, or about 97%, or about 98% identity thereto.

31. The method of any one of claims 1-29, wherein the GM-CSF has an amino acid sequence of SEQ ID NO: 2 or SEQ ID NO: 3, or a variant of about 90%, or about 93%, or about 95%, or about 97%, or about 98% identity thereto.

32. The method of any one of claims 1-29, wherein the GM-CSF is one of molgramostim, sargramostim, and regramostim.

33. The method of claim 32, wherein the GM-CSF is sargramostim.

34. The method of any one of claims 1-33, wherein the GM-CSF is administered at a total dose of about 125 μg, about 150 μg, or about 200 μg, or about 250 μg, or about 300 μg, or about 350 μg.

35. The method of claim 34, wherein the GM-CSF is administered at a total dose of about 250 μg.

36. The method of any one of claims 1-33, wherein the GM-CSF is administered at a dose of about 125 μg, about 150 μg, or about 200 μg, or about 250 μg, or about 300 μg, or about 350 μg.

37. The method of any one of claims 34-36, wherein the GM-CSF is administered twice daily.

38. The method of claim 37, wherein the GM-CSF is sargramostim, administered at a dose of about 125 μg, twice daily.

39. The method of any one of claims 1-38, wherein the GM-CSF is administered via an intravenous route.

40. The method of any one of claims 1-38, wherein the GM-CSF is administered to the lung.

41. The method of claim 40, wherein the GM-CSF is administered via aerosol or nebulizer.

42. The method of claim 41, wherein the aerosol or nebulizer is selected from liquid nebulization, dry powder dispersion and meter-dose administration.

43. The method of any one of claims 1-38, wherein the GM-CSF is administered by inhalation.

44. The method of any one of claims 1-43, wherein the method further comprises administering one or more additional therapeutic agents, selected from remdesivir; favipiravir; galidesivir; prezcobix; lopinavir; and/or ritonavir; and/or arbidol lopinavir/ritonavir; and/or ribavirin; and/or IFN-beta; xiyanping; anti-VEGF-A; fingolimod; carrimycin; hydroxychloroquine; darunavir and cobicistat; methylprednisolone; brilacidin; leronlimab; thalidomide, bamlanivimab, casirivimab, and imdevimab.

45. A method for treating an infection with a coronavirus, comprising:
(a) selecting a patient having an infection with a coronavirus and one or more of
(i) reduced numbers of eosinophils relative to an uninfected state;
(ii) elevated level of ferritin relative to an uninfected state; and/or
(iii) elevated level of CRP relative to an uninfected state and
(b) administering an effective amount of a composition comprising GM-CSF to the patient.

46. The method of claim 45, wherein the method further comprises the step of monitoring eosinophil numbers during the course of treatment.

47. The method of claim 46, wherein an increased number of eosinophil directs continued administration of GM-CSF.

48. The method of claim 46, wherein decreased number of eosinophil directs discontinuation of administration of GM-CSF.

49. The method of claim 45, wherein the method further comprises the step of monitoring the level of ferritin during the course of treatment.

50. The method of claim 49, wherein a decreased level of ferritin directs continued administration of GM-CSF.

51. The method of claim 49, wherein an increased level of ferritin directs discontinuation of administration of GM-CSF.

52. The method of claim 45, wherein the method further comprises the step of monitoring the level of CRP during the course of treatment.

53. The method of claim 52, wherein a decreased level of CRP directs continued administration of GM-CSF.

54. The method of claim 52, wherein an increased level of CRP directs discontinuation of administration of GM-CSF.

55. The method of any one of claims 45-54, wherein the number of eosinophils, level of ferritin, and/or level of CRP is assayed in a biological sample from the patient.

56. The method of any one of claims 45-55, wherein the biological sample comprises blood, respiratory fluid, saliva, or stool.

57. The method of claim 56, wherein the respiratory fluid is from an oropharyngeal (OP) or nasopharyngeal (NP) swab.

58. The method of claim 56, wherein the respiratory fluid is lavage fluid, optionally wherein the lavage fluid comprises a bronchial washing.

59. The method of claim 56, wherein the respiratory fluid is sputum.

60. The method of claim 56, wherein the respiratory fluid is a nasal secretion.

61. The method of claim 56, wherein the respiratory fluid is saliva.

62. The method of any one of claims 45-61, wherein the coronavirus is selected from
(i) a betacoronavirus, optionally selected from severe acute respiratory syndrome coronavirus 2 (SARS-CoV-2), SARS-CoV, Middle East respiratory syndrome-corona virus (MERS-CoV), HCoV-HKU1, and HCoV-0043 and
(ii) an alphacoronavirus, optionally selected from HCoV-NL63 and HCoV-229E.

63. The method of claim 62, wherein the coronavirus is SARS-CoV-2.

64. The method of claim 63, wherein the patient is afflicted with COVID-19.

65. The method of any one of claims 62-64, wherein the patient is afflicted with one or more of fever, cough, shortness of breath, diarrhea, upper respiratory symptoms, lower respiratory symptoms, pneumonia, and acute respiratory syndrome.

66. The method of any one of claims 62-65, wherein the patient is hypoxic.

67. The method of any one of claims 62-66, wherein the patient is afflicted with respiratory distress.

68. The method of any one of claims 62-67, wherein the method prevents or mitigates development of acute respiratory distress syndrome (ARDS) in the patient.

69. The method of any one of claims 62-68, wherein the method improves oxygenation in the patient.

70. The method of any one of claims 62-69, wherein the method prevents or mitigates a transition from respiratory distress to cytokine imbalance in the patient.

71. The method of any one of claims 62-70, wherein the method reverses or prevents a cytokine storm.

72. The method of claim 71, wherein the method reverses or prevents a cytokine storm in the lungs or systemically.

73. The method of claim 72, wherein the cytokine storm is selected from one or more of systemic inflammatory response syndrome, cytokine release syndrome, macrophage activation syndrome, and hemophagocytic lymphohistiocytosis.

74. The method of any one of claims 71-73, wherein the method reverses or prevents excessive production of one or inflammatory cytokines.

75. The method of claim 74, wherein the inflammatory cytokine is one or more of IL-6, IL-1, IL-2, IL-1 receptor antagonist (IL-1ra), IL-2ra, IL-10, IL-18, TNFα, interferon-γ, CXCL10, and CCL7.

76. The method of any one of claims 62-75, wherein the method causes a decrease in viral load in the patient relative to before treatment.

77. The method of any one of claims 62-76, wherein the GM-CSF has an amino acid sequence of SEQ ID NO: 1, or a variant of about 90%, or about 93%, or about 95%, or about 97%, or about 98% identity thereto.

78. The method of any one of claims 62-76, wherein the GM-CSF has an amino acid sequence of SEQ ID NO: 2 or SEQ ID NO: 3, or a variant of about 90%, or about 93%, or about 95%, or about 97%, or about 98% identity thereto.

79. The method of any one of claims 62-76, wherein the GM-CSF is one of molgramostim, sargramostim, and regramostim.

80. The method of claim 79, wherein the GM-CSF is sargramostim.

81. The method of any one of claims 62-80, wherein the GM-CSF is administered at a total dose of about 125 μg, about 150 μg, or about 200 μg, or about 250 μg, or about 300 μg, or about 350 μg.

82. The method of claim 81, wherein the GM-CSF is administered at a total dose of about 250 μg.

83. The method of any one of claims 62-80, wherein the GM-CSF is administered at a dose of about 125 μg, about 150 μg, or about 200 μg, or about 250 μg, or about 300 μg, or about 350 μg.

84. The method of any one of claims 62-83, wherein the GM-CSF is administered twice daily.

85. The method of claim 84, wherein the GM-CSF is sargramostim, administered at a dose of about 125 μg, twice daily.

86. The method of any one of claims 62-85, wherein the GM-CSF is administered via an intravenous route.

87. The method of any one of claims 62-85, wherein the GM-CSF is administered to the lung.

88. The method of claim 87, wherein the GM-CSF is administered via aerosol or nebulizer.

89. The method of claim 88, wherein the aerosol or nebulizer is selected from liquid nebulization, dry powder dispersion and meter-dose administration.

90. The method of any one of claims 62-89, wherein the GM-CSF is administered by inhalation.

91. The method of any one of claims 62-90, wherein the method further comprises administering one or more additional therapeutic agents, selected from remdesivir; favipiravir; galidesivir; prezcobix; lopinavir; and/or ritonavir; and/or arbidol lopinavir/ritonavir; and/or ribavirin; and/or IFN-beta; xiyanping; anti-VEGF-A; fingolimod; carrimycin; hydroxychloroquine; darunavir and cobicistat; methylprednisolone; brilacidin; leronlimab; thalidomide, bamlanivimab, casirivimab, and imdevimab.


SEQUENCE LISTING (3 sequences)

SEQ ID NO: 1 - protein, 127 aa, Artificial Sequence
  Synthetic polymer.
APARSPSPSTQPWEHVNAIQEAPRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 2 - protein, 127 aa, Artificial Sequence
  Synthetic polymer.
APARSPSPSTQPWEHVNAIQEALRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 3 - protein, 127 aa, Artificial Sequence [sequence omitted]